(19) United States
(12) Patent Application Publication
(10) Pub. No.: US 2015/0135423 A1
(43) Pub. Date: May 21, 2015

(54) External Voiding Apparatus and System

(71) Applicants: Isabella K. Sharpe, Jacksonville, FL (US); Eric P. Stord, Melbourne, FL (US)

(72) Inventors: Isabella K. Sharpe, Jacksonville, FL (US); Eric P. Stord, Melbourne, FL (US)

(21) Appl. No.: 14/083,409

(22) Filed: Nov. 18, 2013

(51) Int. Cl. A61F 5/455; A61F 5/453

(52) U.S. Cl. CPC A61F 5/455; A61F 5/453
USPC 4/471

(57) ABSTRACT

An external voiding apparatus includes an oval/canoe shaped receiver cup, with an outlet at one end of the receiver cup, a check valve assembly, and a valve seat attached to the receiver. The receiver cup has open top and gradually sloping, walls that converge at the outlet adjacent a lower area of the cup. The valve assembly is disposed between the receiver cup on the valve seat in the reservoir. The cheek valve may comprise a valve body with an aperture covered downstream by an easily deflectable membrane, or a flat tube type valve. The fluid coupling assembly includes a coupling from the reservoir for a tube. A fluid collection container (e.g. a Foley bag) may be fluidly coupled to the drain tube connected to the reservoir drain. The gasket and receiver cup may include a phosphorescent polymer to provide a glow in the dark effect, a handle attached to the receiver cup and a removable cover. A wide compressible resilient gasket is provided on the top edge of the receiver, which may be anatomically contoured. A support member suitable for holding the aforesaid apparatus in convenient reach of the user and/or associated care givers completes the system of the invention.

### CROSS-REFERENCE TO RELATED APPLICATIONS

This application is a continuation-in-part of parent application Ser. No. 12/799,632 titled “External Voiding Apparatus and System” filed Apr. 28, 2010, the same being incorporated herein by reference and claims priority thereto.

### BACKGROUND

This invention relates generally to urinals, and more particularly, to an external voiding apparatus and system suitable for capturing urine from female and male users, in both clinical and emergency settings. Various devices have been developed for collecting urine from bedridden female and male patients. Such devices typically comprise a container with an inlet configured to channel the flow of urine to a container. By way of example, US Publication No. 2006/0155214 discloses a urine collection device with a funnel connected to a container by a hose. U.S. Pat. Nos. 3,927,426 and 4,270,231 disclose urinals with hinged closures. U.S. Pat. No. 4,568,339 discloses an hourglass shaped funnel with a deflectable rim and an optional gasket. U.S. Pat. No. 4,764,991 discloses a flexible funnel connected to a container with a hose. U.S. Pat. No. 4,771,484 discloses a rigid flume surrounded by a flexible funnel sleeve connected to a hose. U.S. Pat. No. 4,936,838 discloses an hourglass shaped funnel with an annular rim that contacts the wearer. A longitudinal ridge extends from the funnel between the buttocks of the user. U.S. Pat. No. 5,295,983 discloses a urinary collector worn under clothing and undergarments to receive urinary discharges and snugly fits directly around a woman's vulva and urethral meatus. The collector is formed of soft, flexible water-proof material and contains absorbent hydrophobic material and a discharge outlet. U.S. Pat. No. 5,592,699 discloses a mouth piece for attachment to the mouth of a urinal. The mouth piece includes a rim for sealing contact with a user. A liquid barrier or bridge separates the urinal opening into two areas, one of which is closed off and includes a valve only permitting flow of liquid into the urinal. U.S. Pat. No. 5,893,176 discloses a funnel with an inner sealing ring adapted to surround and sealingly engage the exterior of the labia minora and an outer sealing ring adapted to surround and sealingly engage the exterior of the labia majora. U.S. Pat. No. 5,956,782 discloses a urinal device for use by women while seated in a chair or wheelchair. The urinal device has a triangular (i.e., pie-slice) shaped shallow pan with a swollen, bulbous cross-section rim and a perforated splash plate. A handle is affixed to the pan opposite the narrow end of the pan so that the user may insert the pan beneath her body. A drainage hose leads from an outlet to a container. U.S. Pat. No. 6,041,448 discloses an evacuator with a triangular shape. U.S. Pat. No. 6,723,078 discloses a urinal kit with a pair of specifically adapted male and female collector funnels. The male collector funnel forms a cylindrical flexible membrane that has a rollable sidewall and is in fluid communication with a drain tube for connection with a collection conduit. The female collector funnel forms a rigid sidewall terminating at the upper end in an upper sealing ridge forming a contoured upper edge specifically adapted to seal against the user's body when pressed against the user's groin, and is in fluid communication with a drain tube for connection with a collection conduit. U.S. Pat. No. 6,944,587 discloses a urinal with a generally triangular shape for stability. A handle helps users properly position the urinal in a manner that forms a good seal against the relevant portion of the female anatomy.

While the prior art urinals may be effective for their intended purpose, they have shortcomings. Specifically, the aforementioned devices attempt to make waterproof seals with the users, this device is not designed with that intent. This device is intended to be used with minimal contact with the genitals, the female device is extra-labial and the male device requires minimal contact with the glans penis area, whether crowned by foreskin or not. The prior art devices are also not optimally configured to capture and redirect the flow of urine discharged from a female, and male, urethra into a container without appreciable backsplash or leakage. In addition this device can be used for either a female or male user depending on which gasket is attached. Furthermore, the prior art devices are not adapted to connect to Foley collection bags, which are widely available and commonly used for collection of urine discharged from Foley catheters.

The invention is directed to overcoming one or more of the problems and solving one or more of the needs as set forth above.

### SUMMARY OF THE INVENTION

To solve one or more of the problems set forth above, in an exemplary implementation of the basic apparatus of the invention, an external voiding apparatus includes a gasket, an oval/canoe shaped receiver, an outlet on an end of the receiver where a quarter turn external threads are located, a reservoir mounted to the outlet of the receiver, and a check valve assembly that is mounted on a valve seat between the receiver and reservoir. The valve assembly is disposed between the receiver and the valve seat area of the reservoir. The reservoir may contain a stemmed threaded drain with cap of the approximate size to permit a fluid coupling to a tube allowing drainage of the reservoir. The fluid can either be drained to a location away from the user in an emergency setting or to a fluid collection container (e.g. a Foley bag) which is preferably fluidly coupled via the other end of the tube. In addition, in its more complete system embodiments, the invention includes means for storing and implementing the invention that allow it to be easily and conveniently used in both home and institutional settings.

The receiver has an open top or side with gradually sloping side walls that rise to and converge on the outlet located adjacent the lower area of the receiver where the valve and reservoir are connected. The receiver has a length between 4 inches and 8 inches, a width between 2 inches and 6 inches, and a depth between 1 inches and 6 inches. Mounted to the open end of the receiver is the gasket which provides the sealing area to the user. Optionally, the gasket may include a phosphorescent polymer to provide a glow in the dark effect. Optionally, a handle forms part of the receiver for ease of handling by a single user, but can also be detachable. A removable cover may be provided to temporarily cover the gasket side of the receiver/gasket assembly when used as a bed side apparatus. Finally, a support member suitable for holding the aforesaid apparatus in convenient reach of the user and/or associated care givers completes the system of the invention. This support member is preferably implemented by the provision of a support which can be attached to a bedside stand or table (such as a patient hospital table) so as to form the support member. In its preferred embodiments, the system also includes a basket or other similar apparatus which can also be attached to such a bedside stand or table and serves to hold items (such as those useful for the cleaning of the cup and other elements) within ready reach of the user and/or caregiver so as to assist in the implementation of the invention.

A compressible resilient gasket is provided for attachment on the top edge of the receiver. The gaskets are user specific for female and male, either may be attached to the reservoir depending on the gender of the user. The gasket is adapted to conform to the contour of a user's groin when pressure is applied. The gasket, which is thicker than the wall thickness of the cup, defines a rim that overhangs the edges of the cup. The compressible resilient gasket is ideally comprised of an arcuate (e.g., a shallow C-shaped top edge) shaped to conform to the contour of a user.

Likewise, the compressible resilient gasket on the top edge of the cup may also have an arcuate “S”-shaped configuration, shaped to conform to the contour of a user. It may be formed using closed cell foam as well, but this is not presently considered an optimal design as sanitation problems have arisen from the use of such materials for similar purposes in therapeutic settings.

A mechanical connection couples the receiver to the reservoir. The mechanical connection may comprise threads, tabs, and snap fit details.

The check valve assembly includes a valve body with at least one aperture covered by a resilient pliable membrane, or tube with flat pliable tubing attached. The resilient pliable membrane is configured to deflect when urged by discharged liquid. Deflection allows the discharged liquid to travel through the at least one aperture. The membrane may be 5 to 100 mil thick silicone. In the other mention embodiment, the check valve assembly is a flat tube type valve assembly on the apertures. Other appropriate valve assemblies are not excluded from this design.

### BRIEF DESCRIPTION OF THE DRAWINGS

The foregoing and other aspects, objects, features and advantages of the invention will become better understood with reference to the following description, appended claims, and accompanying drawings, where:

FIG. 1A provides an exploded perspective view of an exemplary external voiding apparatus with female gasket according to principles of the invention.

FIGS. 1B and 1C provide views of an exemplary female user shaped gasket for an external voiding apparatus according to principles of the invention, with FIG. 1B providing a plan view and FIG. 1C providing a cross sectional view taken through A-A of FIG. 1B.

FIGS. 1D and 1E provide views of an exemplary male users gasket for an external voiding apparatus according to principles of the invention, with FIG. 1D providing a plan view and FIG. 1 providing a cross sectional view taken through B-B of FIG. 1D.

FIGS. 2A and 2B provide perspective views from opposite sides of an exemplary deflectable membrane type flap valve assembly for an external voiding apparatus according to principles of the invention.

FIGS. 3A and 3B provide perspective views from opposite sides of a flat tube type valve assembly for an external voiding apparatus according to principles of the invention.

FIGS. 4A and 4B provide exploded perspective views from opposite sides of the exemplary deflectable membrane type flap valve assembly for an external voiding apparatus according to principles of the invention.

FIGS. 5A and 5B provide exploded perspective views from opposite sides of the exemplary flat with type valve assembly for an external voiding apparatus according to principles of the invention.

FIGS. 6A and 6B provide views of an exemplary assembled external voiding apparatus with a handle and the female user gasket according to principles of the invention, with FIG. 6A providing a plan view and FIG. 6B providing a side view.

FIGS. 7A and 7B provide views of an exemplary assembled external voiding apparatus with a different type of handle and the male gasket according to principles of the invention, with FIG. 7A providing a plan view and FIG. 7B providing a side view.

FIG. 8 provides an exploded perspective view of an exemplary external voiding apparatus according to principles of the invention with female user gasket; and

FIG. 9 provides a second exploded perspective view of an exemplary external voiding apparatus according to principles of the invention with male user gasket; and

FIGS. 10A through 10D provide views illustrating use of an exemplary assembled external voiding apparatus in relation to female and male genitalia according to principles of the invention, with FIG. 10A providing a view of female genitalia, including a urethral opening, FIG. 10B providing a view of the apparatus placed for use in relation to said female genitalia, FIG. 10C providing a view of male genitalia, and FIG. 10B providing a view of the apparatus placed for use in relation to said male genitalia.

FIG. 11 illustrates a Foley bag alongside a bed to which the tube of the exemplary external voiding apparatus may connect.

FIG. 12 provides a perspective view of a support member for holding and supporting the external voiding apparatus and associated items.

FIG. 13 provides a perspective view of an exemplary detachable handle according to principles of the invention.

Those skilled in the art will appreciate that the figures are not intended to be drawn to any particular scale; nor are the figures intended to illustrate every embodiment of the invention. The invention is not limited to the exemplary embodiments depicted in the figures or the type of valve assembly, handle configuration, shapes, relative sizes, ornamental aspects or proportions shown in the figures.

### DETAILED DESCRIPTION

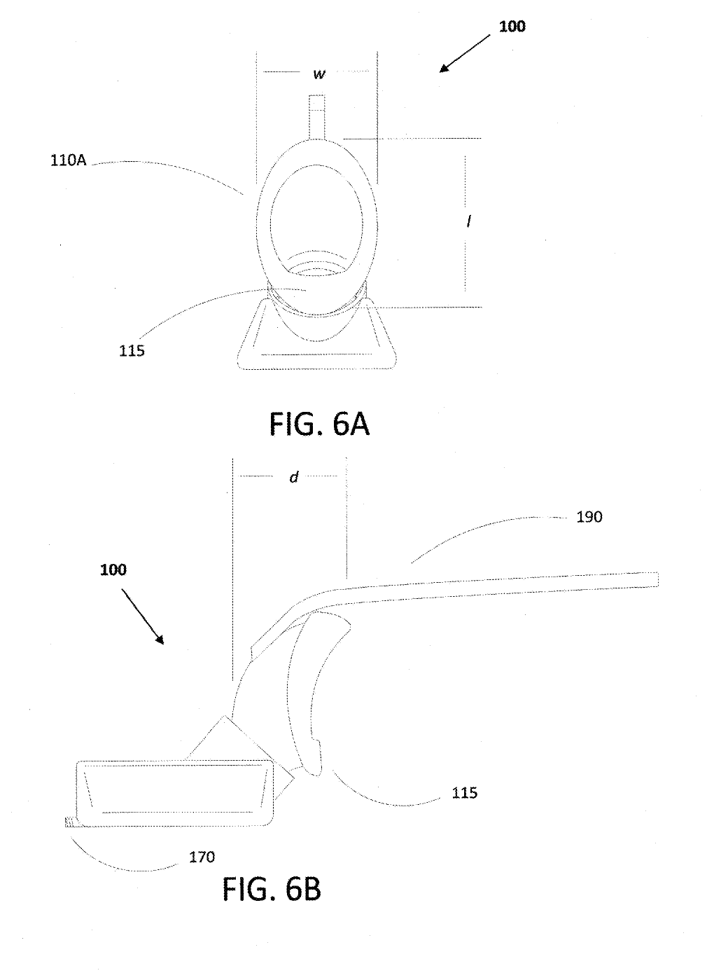

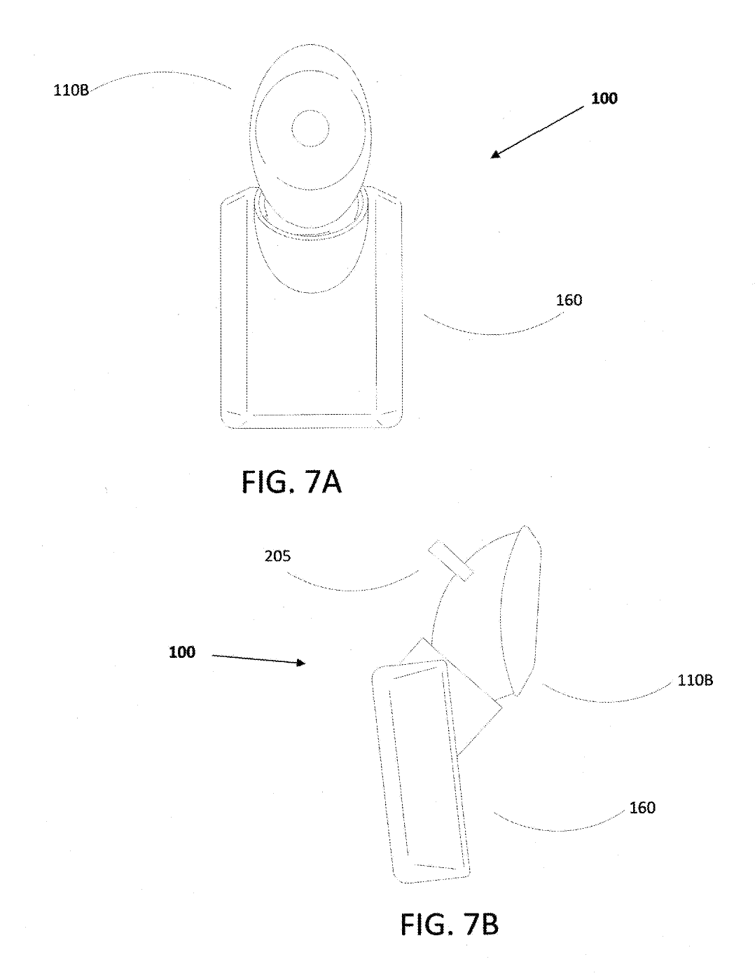

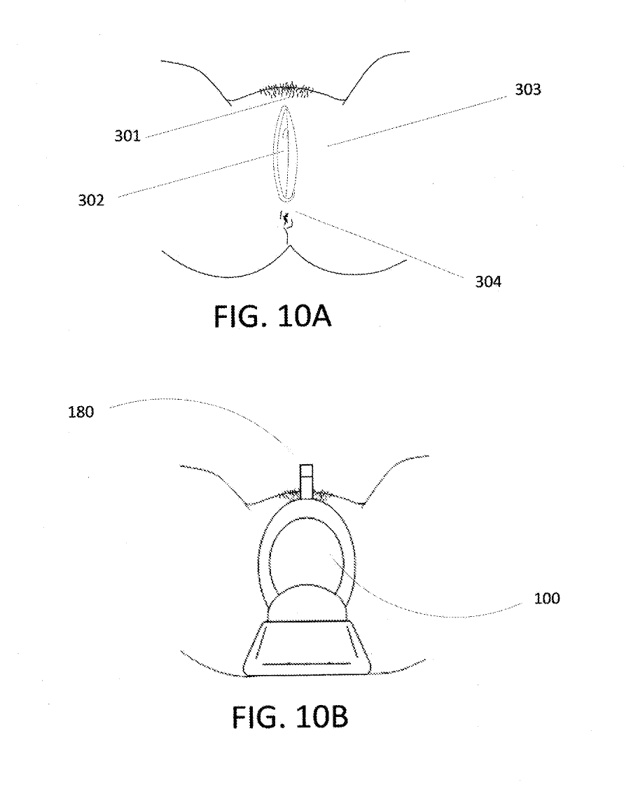

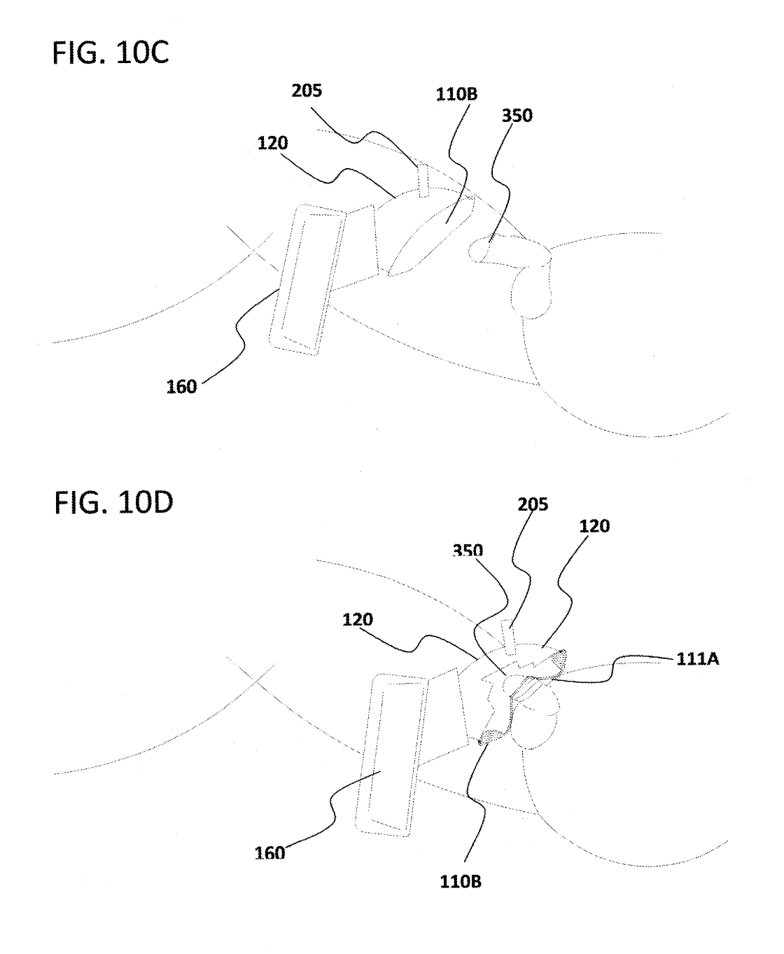

Referring to the Figures, in which like parts are indicated with the same reference numerals, various views of an exemplary external voiding apparatus 100 according to principles of the invention are shown. The exemplary apparatus includes a gender specific gasket 110A (for females) and 110B (for females), a generally oval receiver cup 120 having a receiver outlet 130 with attachment features 130A, a valve assembly 151, 140, and a valve seat 150 area formed in the reservoir 160 (a threaded drain connection may be provided from reservoir 160 to a narrower diameter tube). It should be noted that reservoir 160 as shown is not the only shape or size that could be available. Also other orientations of the reservoir to the receiver are possible, as are other orientations of the gasket 110 to the receiver 120 (e.g. top/bottom reversal), to facilitate usage of device. Some of the other orientations are shown in the illustrations. It should particularly be noted that the orientation of the reservoir 160 to the receiver cup 120 shown in FIGS. 6A, 6B, and 10B, is (as illustrated) particularly suited for use with a female, especially a prone female (though other orientations are possible). Likewise, the orientation illustrated in FIGS. 7A, 7B, 10C and 10D, is more generally suited for use with males (though once again other orientations are possible). Thus, the ability to change orientation between reservoir 160 and receiver cup 120 should at least comprehend the 180 degree change illustrated in these drawing figures.

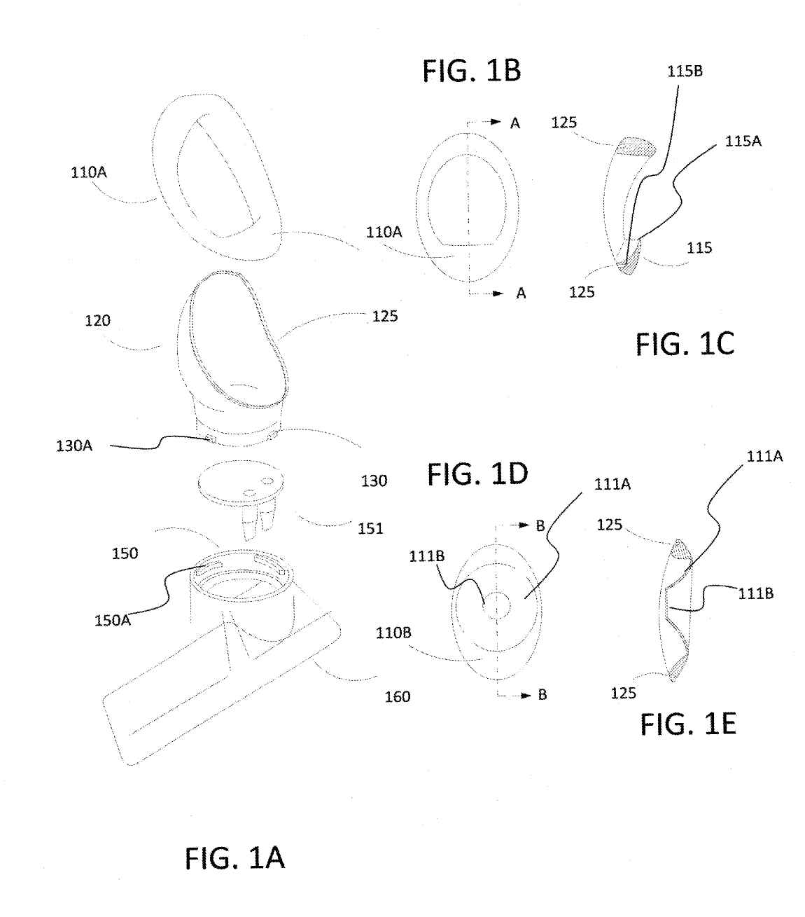

The receiver 120 (bordered by gasket 110) receives urine discharged from a urethral opening and guides the mine through receiver 120 and outlet 130. As best seen in FIG. 1C, the lower part of the female version 110A of the gasket 110, serves as a dam 115. The dam area 115 is reinforced to provide a more rigid structural area to exert force on the perineum 304 or on portions of the female genitalia 302 below the female urethra thereof when the device is pressed into position so as to prevent urine from traveling in the labia, or elsewhere, below the female version of the gasket 110A of the device. (See, e.g., FIGS. 10A and 10B; however, it should be noted that the function of the female version is not dependent in any way on whether the labia are open—as shown in FIG. 10A—or closed). As will be noted from review of FIGS. 1B and 1C, the dam area 115 of female gasket version 110A has a narrow lip 115A so that when pressed in place against the female genitalia 302 under the urethra 302 it will press in slightly so that genital tissue will overhang lip 115A and urine will be caught and directed into receiver cup 120 by undercut 115B. The receiver cup 120 includes an opening with gradually sloped walls that converge at the outlet 130 at the lower end of the receiver 120. The shape of the cup 120 acts as a funnel, capturing and redirecting a stream of discharged urine towards the outlet 130 without excessive splash-back, and is suitable for use with males as well as females.

The receiver cup 120, and associated gaskets 110A, 110B, may come in various sizes to accommodate a range of male and female users. In a preferred embodiment the cup 120 with gasket 110A, is sized to cover an adult female's vulva. In an exemplary embodiment, the length, l, as shown in FIG. 6A, is between 4 inches and 8 inches, preferably about 6 inches and is sized to: (1) stretch from and snugly fit along the arch of the pubic bone or mons pubis 301 at its upper end above the vulva (female genitalia 302); stretch along and snugly fit beside both sides of the female genitalia 302 in the inguinal folds 303 between the legs and female genitalia 302; and (3) reach its lower apex adjacent outlet 115 where it fits below female genitalia 302 at or around perineum 304. In the exemplary embodiment, the width, w, is between 2 inches and 6 inches, preferably about 4 inches. In the exemplary embodiment, the depth, d, as shown in FIG. 6B, is between 1 inch and 6 inches, preferably about 3 to 4 inches.

The receiver cup 120 is comprised of a substantially rigid material to support the reservoir 160, and the gasket 110 which is suitable for contact with human flesh and urine. Although any of a variety of metals, plastics and composites may be used, a plastic cup 120 is preferred. In a particular exemplary implementation, the receiver cup 120 is primarily comprised of a rigid plastic or polymeric material, such as polyvinyl chloride (PVC), polyethylene, polypropylene, polystyrene, acrylics, cellulosics, acrylonitrile-butadiene-styrene terpolymers, urethanes, thermo-plastic resins, thermo-plastic elastomers (TPE), acetal resins, polyamides, polycarbonates and polyesters. While many other materials may be used alone or in combination with the aforementioned materials and/or other materials, without departing from the scope of the present invention, preferably the material is relatively inexpensive, easy to use in manufacturing operations and results in an aesthetically acceptable, durable product. The gasket 110 may be made of similar material as to facilitate comfortable contact with the user. The material may further include additives to provide desired properties such as desired colors, structural characteristics, glow-in-the dark properties and thermal reactivity (e.g., color changes according to heat).

By way of example and not limitation, a phosphorescent polymer additive, such as aluminate based phosphors, may be added to some or all parts of the gasket 110 and receiver cup 120 to provide a glow in the dark effect (i.e., to adsorb light energy and continue to release that energy as visible light after the energy source is removed). Advantageously, such an embodiment provides a device that is easy to locate in darkened conditions, making the device easy to locate at nighttime.

The receiver cup 120 and its components may be produced using any suitable manufacturing techniques known in the art for the chosen material, such as (for example) injection, compression, structural foam, blow, or transfer molding; polyurethane foam processing techniques; vacuum forming; casting and milling. Preferably, the manufacturing technique is suitable for mass production at relatively low cost per unit, and results in an aesthetically acceptable product with a consistent acceptable quality.

The compressible resilient gasket 110 covers the top edge of the cup 120. The compressibility of the gasket 110 allows the gasket 110 to conform to the contour of the user's groin when pressure is applied. When pressure is released, the resilient gasket returns to its original shape. The gasket 110 may be attached to the receiver cup via a formed slot and bead arrangement 125, and is removable and washable.

The thickness and resiliency of the gasket 110 allow sufficient deformation to accommodate a wide range of body contours. The height, may be between ¼ inch and 2 inches, preferably about 1 inch.

As shown in FIGS. 1B through 1E, the gasket 110 (A or B) is wider than the sidewall of the receiver cup mounts 125. Therefore, the gasket provides a rim that prevents liquid traveling up the sidewall of the receiver cup 120 from being discharged past the edge of the cup 120.

The gasket may have various additional cross sectional shapes for the area that comes in direct contact with the user. An ellipsoid C-shaped cross-section or S-shaped cross-section may be employed to assist in preventing back splash. D-shaped, E-shaped, H-shaped, O-shaped and U-shaped gasket profiles are also possible. These and other shapes may be used within the spirit and scope of the invention.

In the preferred embodiment, the gasket 110, and/or the receiver cup 120 may have a contour that is curved to conform to the anatomy of a user. Such a curved contour is illustrated in the drawing figures. This device is designed to be manufactured in multiple sizes, which will dictate different dimensions. The curvature for the female version of the gasket 110A follows the contours of the mons pubis 301 and inguinal folds 303 as described above. The curvature, as seen from a side view, generally resembles a shallow C or a parenthesis, with the fore and aft portions protruding from the cup further than the intermediate portions. It may also be bowed slightly inwardly or outwardly at the sides to better fit into inguinal folds 303 or at its upper end to better fit along the pubic arch 301.

The gasket 110 may be formed from any elastomeric material suitable for constructing a soft, compressible, resilient gasket structure that will contact human tissue and urine. The gasket 110 and its components may be produced using any suitable manufacturing techniques known in the art for the chosen material, such as (for example) extrusion, casting, polyurethane foam processing techniques, casting and milling. Preferably, the manufacturing technique is suitable for mass production at relatively low cost per unit, and results in an aesthetically acceptable product with a consistent acceptable quality.

In use, the gasket creates a semi-seal (e.g., semi-watertight). The gasket also provides a comfortable surface against the user. However, the device 100 does not primarily depend on this seal to accomplish its purposes. Aside from the other features previously described, the shape of the dam 115 across the bottom of the female version 110A provides a narrow lip 115A that helps to guide and direct any urine that might trickle downward from the urethra into the receiver 120 when pressed into position. Likewise, the diaphragm 111A and aperture 111B combination of the male version 110B, provides a means for assuring that urine from the male user is also appropriately directed and captured.

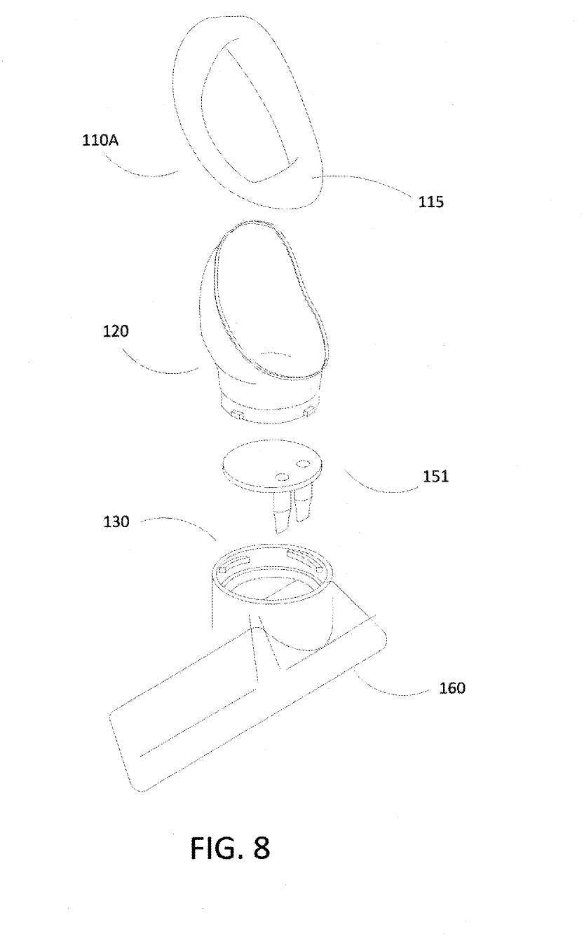

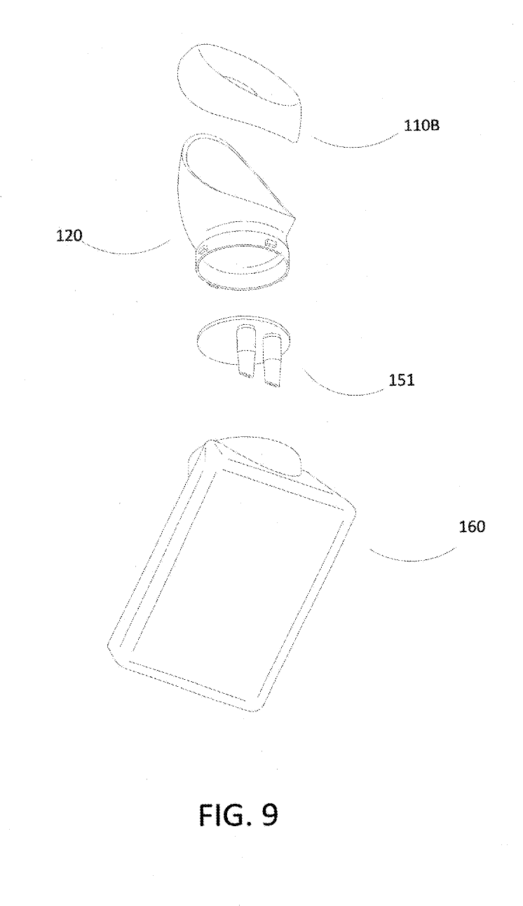

No matter which gasket version 110A, 110B is used, urine received in the receiver cup 120 is discharged through receiver cup outlet 130, which is an aperture in the receiver cup 120, through which liquid may freely flow to and through a valve 151, 140. Liquid flowing through the cup outlet 130 transits valve 151, 140 past attachment features 130A at outlet 130. The attachment features 130A at outlet 130 are chosen to enable interlocking fluid-tight engagement with mating reservoir features 150A of reservoir 160 (which may also he fluidly coupled to a drain hose). The attachment features 130A, 150A at the juncture of outlet 130 and reservoir 160 may comprise threads, tabs, snap fit details, or other structures suitable for releasable, fluid tight engagement with reservoir 160. Such features may, for example, comprise a quarter turn male thread used to attach a mating receptacle having female quarter turn threads as illustrated. (See, e.g., FIGS. 1A, 8, and 9). This has the advantage of allowing the reservoir 160 to be locked into position at various positions at 90 degree intervals so as to allow four stable juncture positions/orientations between the reservoir 160 and cup 120 with different positions being suitable and/or convenient for different uses. (Cf., e.g., the different positions illustrated in FIGS, 6A through 7B for male/female users).

A check valve assembly 151, 140 maintains the fluid flow in a single direction. The check valve assembly 151, 140 is disposed between the receiver outlet 130 and the valve seat area 150 on the reservoir 160. The valve assembly 151 140 allows fluid to flow from the outlet 130 through and into the reservoir 160. The valve assembly 151, 140 restricts or prevents flow in the opposite direction. Thus, the valve assembly 151, 140 helps retain discharged liquids and associated odors within the reservoir 160, thereby preventing or limiting spillage and seepage when the device is moved after usage.

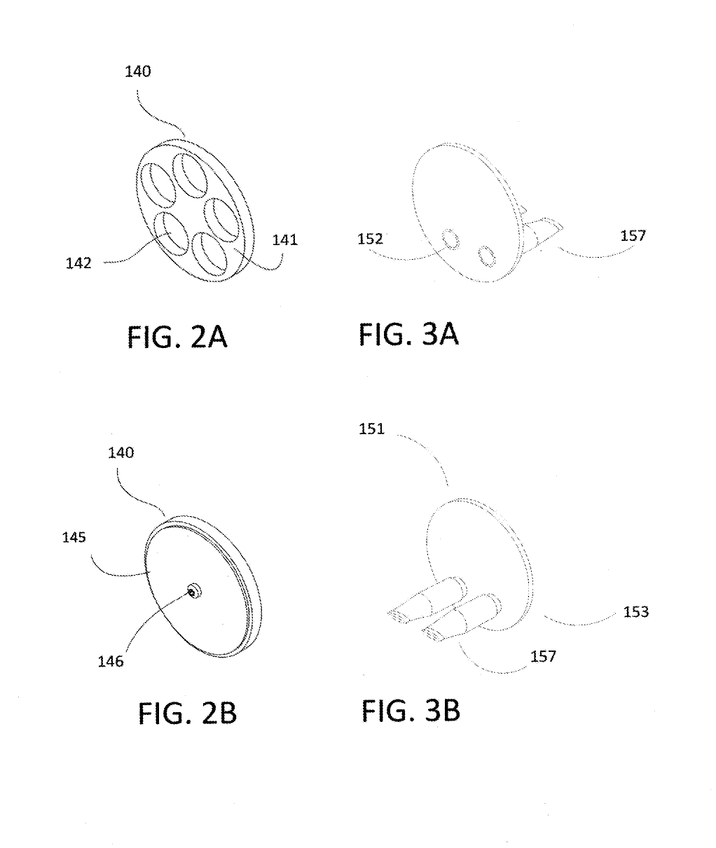

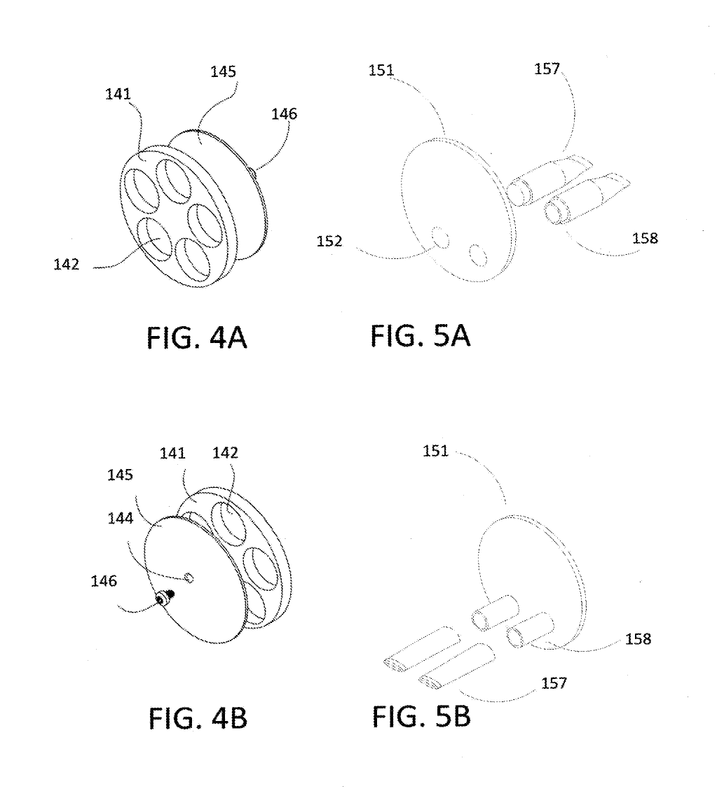

Illustrative examples are provided in drawing FIGS. 2A, 2B, 4A and 4B for a flap valve assembly 140 and in FIGS. 3A, 3B, 5A, and 5B for tube style valve assemblies 151. The valve assemblies may be flap style valve assemblies 140 that include a thin pliable resilient membrane 145 covering one or more apertures 142 in a valve body 140. In FIGS. 2A, 2B, 4A and 4B, liquid flows through the one or more apertures 142 and impacts the membrane 145, causing the membrane to deflect. The deflection allows the liquid to flow past the membrane and continue its travel. A fastener, such as a screw 146, secures the membrane 145 at one point 144, to the valve body 141.

The membrane 145 may be formed from any elastomeric material suitable for constructing a thin, resilient pliable sheet that will contact human urine. In an exemplary embodiment, a thin silicone sheet is used. However, the invention is not limited to silicone. Other thin pliable resilient materials may be utilized without departing from the scope of the invention. The thickness of the membrane is not particularly important, so long as it retains resiliency, provides a seal and yields under the pressure of discharged urine. Thickness will, also depend on the modulus of the material, with thickness decreasing as modulus increases. Modulus will depend in part upon the composition and manufacturing and environmental conditions. By way of example and not limitation, a 5 to 100 mil thick silicone rubber membrane may be used.

Alternatively, a flat tube type of valve assembly 151 may be manufactured from rubber or synthetic elastomer. One end of the flat tube 157 valve is stretched over the outlet tubes 158 mounted on the valve body 151, conforming itself to the shape of the outlet tubes 158. The other end of the flat tube 157 retains its natural flattened shape. When a fluid is discharged through the valve, the flattened end opens to permit the fluid to pass. When pressure is removed, the flat tube end returns to its flattened shape, preventing backflow.

The valve assembly 151, 140, sits in the valve seat 150, abutting the reservoir 160 and the receiver outlet 130. During assembly, the valve assembly is seated and the throat 130 is positioned on the valve seat 150 with the engagement features of 130 and 160 aligned for engagement. Engagement may be achieved by twisting and or snap-fit action. When engaged, the edge of the receiver outlet 130 is secured against the valve assembly 151, 140, which is sandwiched between the receiver outlet 130 and the reservoir 160 on the seat 150. One or more o-rings or gaskets may be used to ensure a fluid-tight seal. An exemplary assembled apparatus is shown in FIGS. 6A and 6B (and in FIGS. 7A and 7B).

The invention is not, however, limited to the valve assemblies shown in the drawing figures. The number and arrangement of apertures may vary. The shape and features of the valve body 141 and 151, membrane 145, and tubes 147 may also vary. Other types of check valve assemblies may be used in lieu of or in addition to the valve assemblies shown in FIGS. 2A through 5B.

Valve seat 150 resides upstream of the reservoir 160 and may be integrally formed with the reservoir 160. The reservoir 160 accumulates and temporarily stores liquid discharged through the valve assembly 151. The reservoir 160 provides a temporary collection compartment until the collected liquid is completely discharged through the optional drain 170 through a tube into a collecting bag or by detaching the reservoir from the receiver and empting into another container. The reservoir 160 may be sized to hold an entire volume of urine discharged from a user or hold only a portion of the urine while drainage through the drain 170 takes place.

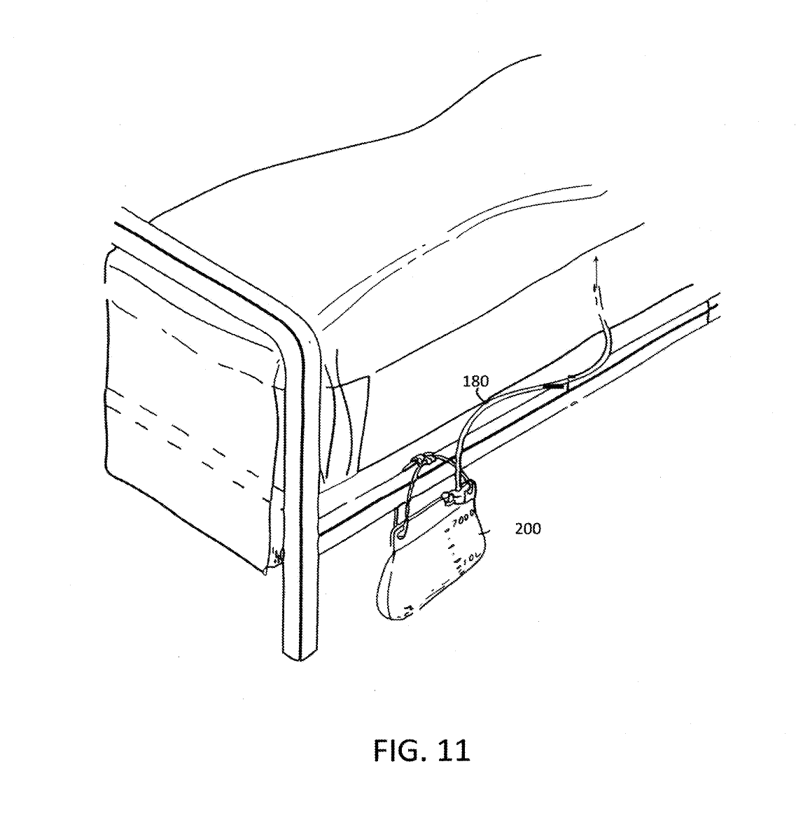

While only the drain 170 is shown in FIG. 6B, persons skilled in the art will appreciate that a tube 180 connected to drain 170 will have a length and he equipped with a coupling for attachment to a urine collection device 200, such as a Foley bag, as conceptually illustrated in FIG. 11. The drain tube 180 may connect directly to the bag 200 or to a tube connected to the bag 200. Any suitable leak-free coupling may be utilized, including, but not limited to, Luer-Lok® or Luer-Slip® (trademark of Becton, Dickinson and Company Corporation, New Jersey) style fittings or the like.

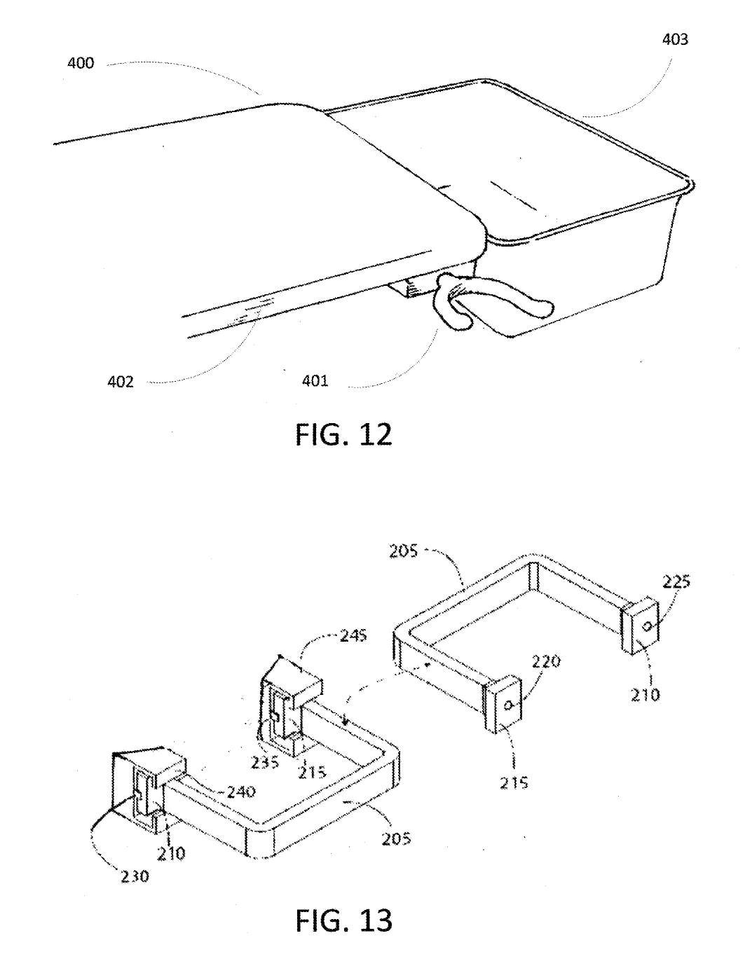

A support member 400 suitable for holding the aforesaid apparatus in convenient reach of the user and/or associated care givers completes the system of the invention. This support member 400 is preferably implemented by the provision of a reservoir cup support. 401 which can be attached to a bedside stand or table 402 (such as a patient hospital table) so as to form the support member. The cup support can, as illustrated in FIG. 12, be such that the external voiding apparatus 100 can easily be placed between said arms and supported. In its preferred embodiments, the system also includes a basket 403 or other similar apparatus which can also be attached to such a bedside stand or table and serves to hold items (such as those useful for the cleaning of the cup and other elements) within ready reach of the user and/or caregiver so as to assist in the implementation of the invention.

In FIG. 6B an embodiment with an optional handle for the female user is shown. The handle 190 facilitates gripping and holding the assembly 100 in place by the female user of the device. Thus, the female user, whether in a prone or upright position, may use the handle 190 to pull the gasket 110A of the device up against and around the vaginal area 302. The handle 190 may also be used to hang the assembly 100 from a bed rail, night table handle, or other support, such as support 401. The handle may be ergonomically configured and fitted or overmolded with a comfortable hand grip. Preferably, as illustrated in the drawing figures, the handle 190 should be positioned horizontally as shown in the drawing figures for ease in gripping the handle by the user (as previously described) and/or by someone giving assistance at the bedside. It facilitates the user and/or care giver in pressing, the assembly 100 firmly into position prior to and during use.

Another embodiment of the removable handle 205 is shown in FIGS. 7A, 7B, and 13. In this embodiment the handle 205 is reasonably attached to the fore section of the receiver cup 120. The handle includes a pair of tabs 210, 215 configured for releasable engagement in corresponding grooves 240, 245 that are molded as part of or otherwise joined to the receiver cup 120. The tabs also include features (e.g., a slight protuberances 210, 215) configured to fit in complementary (e.g., a slight depression) feature in groove 240, 245, to register for locking engagement. Optional stops 230, 235 are also provided on one side of the groove 240, 245 to prevent sliding past the end of the groove 240, 245.

Although use with female vulva/genitalia 301 is illustrated in most of the drawing figures with the gasket version 110A, persons of skill in the art will appreciate that a male person may also use the apparatus by directing the penis towards the inlet of the male gasket 110A. The shape of the gaskets 110 and receiver 120 accommodates various body positions. In each case, the outlet 130 is preferably tilted downward to facilitate the flow of discharged urine under the influence of gravity. However, a more optimally designed male gasket version 110B is also illustrated, and is preferred for usage with men. As best seen in FIGS. 1D, 1E, 7A, 7B, 10C and 10D, version 110B is provided with a diaphragm 111A extending inwardly towards the center of the gasket 111B as well as towards the interior of receiver cup 120, with an aperture 111B located at its approximate center. Diaphragm 111A as well as the periphery of aperture 1118 are relatively flexible, and easily placed as illustrated in FIGS. 10C and 10D to conform with the male genitalia with the head of the penis 350 interfacing with and directed into aperture 111B for the purpose of urination. As will be noted also from these drawing figures (in contrast to FIGS. 10A and 10B) a different orientation of reservoir 160 to receiver cup 120 is also preferred for use with a prone male, and is facilitated by the ability to realign the orientation of the parts named for this purpose.

During use, urine flows from the individual past the gasket 110 and into the receiver cup 120. The gasket 110 limits leakage between the user and the receiver cup 120. The urine is guided by the contour of the cup 120 and the influence of gravity to the outlet of the cup 130, through the check valve 151, and into the reservoir 160. The check valve 151 opens under the pressure of the urine. From the reservoir, urine drains through the drain tube 170. From the drain 170, the urine may travel via a tube 180 attached to a collection container, such as a Foley bag. When urination is complete, the receiver cup 120 may be cleaned and covered using an optional cover. Additionally, while the apparatus is draining into a collection bag, the device 100 may be hung on cup support 401 as described above or by using optional handle 190, or 205.

All mechanical (e.g., threaded) fittings are universal to fit the remaining parts of the apparatus 100 regardless of size. This facilitates maintenance and assembly.

A cover for the open portion of receiver cup 120 may also be provided. The cover may be rigid or flexible. By way of example, a flexible plastic bag-like cover with elastic to attach like a shower cap may be utilized. Alternatively, a molded rigid plastic cover sized to fit over the opening of the gasket 140, may be utilized. The cover may be removed, cleaned, discarded and replaced. The cover prevents any spillage of urine residue and facilitates bedside storage and cleanliness until the apparatus is needed.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

In application of the device is should be noted the gasket 110A (female with dam 115) and 110B (male with circular seal) are both designed to perform their function external to the genitals. In the case of the 110A, the depression of the gasket, and more specifically the dam 115, to the vaginal area below the urethra to the perineum area is apply with adequate pressure to force urine out of the labial folds and into the device. The dam 115 protrudes slightly from gasket 110A, and is structurally reinforced, to aid in the accomplishment of goal. The Male gasket 110B works by a similar pressure and is not designed for the insertion of the penis through the circular seal, but rather to produce a seal around the glans penis, or foreskin in that area as the case may be. The expectation is the urine will flow in the cup and back onto the gasket, with the shape of the gasket directing the urine away from the opening, which is sealed by the penis, and into the receiver cup 120 where it will discharge through the valve assembly as described above. It should be noted the female gasket can be mounted to the receiver with the dam toward the reservoir side of the receiver for use when the user is in a supine position, or away from reservoir when used in a squatted positions. The reservoir may also be mounted in other position to facilitate usage depending on the user position, and the application of the device. Different mounting of the reservoir to the receiver are shown in the illustrations.

What is claimed is:

1. An external voiding apparatus system. comprising:
a substantially oval shaped receiver cup having an upper opening with a surrounding edge and an outlet at a lower area of the cup, said cup having gradually sloped walls that converge from said edge towards said outlet, and said edge having a dorsal portion adapted for placement towards the anus of a user and a ventral portion adapted for placement towards the pubic bone of a user;
an attachment feature throat extending from the outlet of the cup;
a removable reservoir joined to said throat so as to be in fluid communication therewith;
a valve seat proximate the juncture of said throat and said reservoir;
a valve assembly positioned in said valve seat so as to he intermediate said reservoir and said outlet, said valve assembly allowing fluid movement from cup to reservoir and inhibiting fluid movement from reservoir to cup; and
interchangeable compressible resilient gaskets mountable to said cup so as to cover said edge and surround said upper opening, which gaskets are one of adapted for use by a female user urinating into the cup, and adapted for use by a male user urinating into the cup.

2. The system of claim 1, wherein the gasket specifically adapted for use by a female urinating into the cup is provided with a reinforced dam area at its dorsal portion, which dam is adapted for placement dorsally of the female user's urethral opening.

3. The system of claim 2, wherein said dam area is provided with a narrow lip and an undercut.

4. The system of claim 1, wherein the gasket specifically adapted for use by a male urinating into the cup is provided with a flexible diaphragm covering the cup opening with an aperture located therein, said aperture being sized to generally accommodate the end of the penis of a male user.

5. The system of claim 4, wherein said diaphragm is inclined inwardly towards the outlet of the cup.

6. The system of claim 1, wherein said removable reservoir and said cup may be mounted to each other in multiple orientations to provide appropriate orientation for usage.

7. An external voiding apparatus system, comprising:
a substantially oval shaped receiver cup having an upper opening with a surrounding edge and an outlet at a lower area of the cup, said cup having gradually sloped walls that converge from said edge towards said outlet, and said edge having a dorsal portion adapted for placement towards the anus of a user and a ventral portion adapted for placement towards the pubic bone of a user;
an attachment feature throat extending from the outlet of the cup;
a removable reservoir joined to said throat so as to be in fluid communication therewith;
a valve seat proximate the juncture of said throat and said reservoir;
a valve assembly positioned in said valve seat so as to be intermediate said reservoir and said outlet, said valve assembly allowing fluid movement from cup to reservoir and inhibiting fluid movement from reservoir to cup;
interchangeable compressible resilient gaskets mountable to said cup so as to cover said edge and surround said upper opening, which gaskets are at least one of: adapted for use by a female user urinating into the cup, and adapted for use by a male user urinating into the cup;
wherein the gasket specifically adapted for use by a female urinating into the cup is provided with a reinforced dam area at its dorsal portion, which dam is adapted for placement dorsally of the female user's urethral opening; and
wherein the gasket specifically adapted for use by a male urinating into the cup is provided with a flexible diaphragm covering the cup opening with an aperture located therein, said aperture being sized to generally accommodate the end of the penis of a male user.

8. The system of claim 7, wherein at least one of: said dam area is provided with a narrow lip and an undercut, and said diaphragm is inclined inwardly towards the outlet of the cup.

9. The system of claim 8, wherein said removable reservoir and said cup may be mounted to each other in multiple orientations to provide appropriate orientation for usage.

10. The system of claim 1, wherein at least one of:
said cup has a length between 4 inches and 8 inches, a width between 2 inches and 6 inches, and a depth between 1 inches and 6 inches,
said interchangeable gaskets are shaped and contoured to conform to the extra-labial outline of a female user's groin, and
said interchangeable gaskets have a thickness that exceeds a thickness of the top edge of the cup, defining a rim that overhangs the top edge of the cup when said interchangeable gaskets are mounted thereto.

11. The system of claim 9, wherein at least one of:
said cup has a length between 4 inches and 8 inches, a width between 2 inches and 6 inches, and a depth between 1 inches and 6 inches,
said interchangeable gaskets are shaped and contoured to conform to the extra-labial outline of a female user's groin, and
said interchangeable gaskets have a thickness that exceeds a thickness of the top edge of the cup, defining a rim that overhangs the top edge of the cup when said interchangeable gaskets are mounted thereto.

12. The system of claim 1 wherein at least one of:
said valve assembly comprises a valve body with at least one valve body aperture covered by a resilient pliable membrane, said resilient pliable membrane being configured to deflect when urged by discharged liquid, thereby allowing the discharged liquid to travel through the at least one valve body aperture,
said valve assembly comprising a flat tube type valve, and
Said valve assembly includes a resilient pliable membrane of 5 to 100 mil thick silicone.

13. The system of claim 11, wherein at least one of:
said valve assembly comprises a valve body with at least one valve body aperture covered by a resilient pliable membrane, said resilient pliable membrane being configured to deflect when urged by discharged liquid, thereby allowing the discharged liquid to travel through the at least one valve body aperture,
said valve assembly comprising a flat tube type valve, and
Said valve assembly includes a resilient pliable membrane of 5 to 100 mil thick silicone.

14. The system of claim 1, herein said reservoir includes a reservoir outlet that may at least one of be opened to empty the reservoir, and be used to fluidly attach the reservoir for drainage to a foley bag.

15. The system of claim 13, wherein said reservoir includes a reservoir outlet that may at least one of be opened to empty the reservoir, and he used to fluidly attach the reservoir for drainage to a foley bag.

16. The system of claim 1, wherein at least one of: a handle is joined to said cup proximate the ventral portion thereof, a handle is joined to said cup proximate the ventral portion thereof and extends upwardly therefrom, and a handle is detachably joined to said cup proximate the ventral portion thereof.

17. The system of claim 15, wherein at least one of: a handle is joined to said cup proximate the ventral portion thereof, a handle is joined to said cup proximate the ventral portion thereof and extends upwardly therefrom, and a handle is detachably joined to said cup proximate the ventral portion thereof.

18. The system of claim 1, further comprising a support member capable of holding the cup in spaced relationship to a floor, wherein said support member comprises at least one of: a cup support attached to a table, and a basket attached to a table.

19. The system of claim 17, further comprising a support member capable of holding the cup in spaced relationship to a floor, wherein said support member comprises at least one of: a cup support attached to a table, and a basket attached to a table.
